(19) United States
(12) Patent Application Publication
(10) Pub. No.: US 2007/0202137 A1
(43) Pub. Date: Aug. 30, 2007

(54) Device For Sensing Of Motile Living Organisms And Uses Thereof

(76) Inventor: Colin John Ingham, Wageningen (NL)

Correspondence Address: AMSTER, ROTHSTEIN & EBENSTEIN LLP, 90 PARK AVENUE, NEW YORK, NY, 10016, US

(21) Appl. No.: 10/593,652

(22) PCT Filed: Apr. 20, 2005
(86) PCT No.: PCT/EP05/04227

§ 371 Date: Oct. 20, 2006

(30) Foreign Application Priority Data
Apr. 21, 2004 (EP) 04447102.7

(51) Int. Cl. C12M 3/00; A01N 25/00; A61K 9/70

(52) U.S. Cl. 424/405; 424/443; 435/289.1

(57) ABSTRACT

The present invention relates to the field of devices for studying behaviour of motile living organisms in a multiplexed array format. The present invention provides devices comprising a solid porous support having first and second surfaces, said first surface comprising an agent and/or condition delineating behavioural and/or physical barriers for motile living organisms, and, where behavioural, said barriers adapted for sensing by said living organisms and hence forcing said living organisms to remain localized within a predefined region of said support. In addition the invention provides methods for screening test molecules or effector molecules which affect behaviour, motility and/or health of a motile living organism.

### FIELD OF THE INVENTION

The present invention relates to the field of devices for studying behaviour of motile living organisms in a multiplexed array format. In addition the invention relates to methods for screening test molecules or effector molecules which affect behaviour, motility and/or health of a motile living organism. Specifically, the invention relates to an adaptation of methods for high-throughput screening which are currently often performed in a multi-well plate format.

### BACKGROUND TO THE INVENTION

With the advent of combinatorial chemistry approaches to identify pharmacologically useful compounds, it is increasingly evident that there is a need for methods and apparatuses at multiplexed levels, capable of performing high throughput screening (HTS) and characterization of pharmacological profiles and corresponding potencies of the compounds in synthesized combinatorial libraries. The primary function of HTS is to test various chemical compounds from a compound library against multiple disease targets in many different biological assays. Traditional HTS commonly utilizes 96-well microtiter plates. There is a large incentive in the pharmaceutical industry to miniaturize these microplate assays to reduce cost, reduce waste, and speed up timelines. There has been a change from the 96-well format to higher well densities such as 384- and 1536-well formats.

WO 99/35496 provides a method and apparatus for high density format screening for bioactive molecules with a much simplified technique for test compound delivery to a layer of cells, i.e. without the need of complicated fluid handling. In the said method, up to 6144 test compounds may be simultaneously screened for bioactivity.

U.S. Pat. No. 6,103,479 discloses a further example of a microarray of living cells. The disclosed miniaturized cell arrays characterized by a reduced well and array size allow for high content screening. The non-porous character, however, does not allow the cells growing in/on the wells or cell binding sites on the array to overcome spreading under standard growth conditions which obviously interferes with proper cell sample discrimination.

Also WO 03/102578 relates to a method for high throughput cell-based assays using versatile living microarrays consisting of monolayers of cells on porous supports.

These assays and arrays however remain limited for use with cells which form monolayers or islands of cells. Moreover, maintaining multiple cell lines or types of cell cultures is extremely costly and labour intensive.

Whilst, at the one hand improving the high density format screening of bioactive molecules by eliminating the well-concept of the assays and array, these arrays are not suitable for testing motile cells or motile living organisms.

Therefore there is a need for array-based functional biological assays in a multiplexed format allowing a broader range of living cells and organisms to be targeted.

There is also a need for reliable and reproducible screening methods and devices for performing comparative and high throughput analyses on and with living organisms without impairing their freedom of movement by external physical acts.

As will be appreciated in the art, there is a continuous need for improved devices or arrays or supports for use in these methods.

### SUMMARY OF THE INVENTION

The present invention meets the need in the art by providing a device suitable for maintaining and/or rearing motile living organisms in a multiplexed array format.

The device is very versatile and is used for studying behaviour and motility and other characteristics of a variety of motile living organisms in a multiplexed array format for organism normally considered different to handle in this fashion. The device is also used for high throughput screening of the effect of test compounds on these living organisms.

In one embodiment, the invention relates to a device comprising a solid porous support having first and second surfaces, said first surface comprising an agent and/or condition delineating behavioural and/or physical barriers for motile living organisms, and, where behavioural, said barriers adapted for sensing by said living organisms and hence forcing said living organisms to remain localized within a predefined region of said support, wherein said barriers are printed on the first surface of the porous support so that it is drawn into the porous support and as such, completely or in part comprised within the pores of the porous support, therewith forming a three-dimensional compartmentalization of the porous support. Said motile living organisms are not physically immobilized on said solid support. Said agent and/or condition is printed or placed onto the first surface of said device to delineate behavioural and/or physical barriers.

According to a further embodiment, the invention relates to a device as described above, wherein said agent is mixed with a permanent compound or wherein said condition is localized within a permanent compound, said permanent compound being printed or placed on said first surface and constituting a behavioural and/or physical barrier. Preferably, said permanent compound is a polymeric material containing at least one of the following: latex, rubber, plastic, resin, glue, protein or polypeptide or carbohydrate.

The invention also relates to a device as described above, wherein said permanent compound is a non-polymeric material.

The invention specifically relates to any of the devices described above wherein said physical barrier is substantially flat.

According to one embodiment, the invention relates to any of the devices described above, wherein said agent is a repellent, further characterized in that said agent is comprised within said barriers surrounding said predefined region wherein said organism needs to remain.

According to another embodiment, the invention relates to any of the devices described above, wherein said agent is an attractant, further characterized in that said agent is comprised within the predefined region wherein said organism needs to remain.

Preferably, the agent comprised in or on the devices of the invention is chosen from the group consisting of hormones, pheromones, detergents, nutrients including prey organisms or extracts thereof, amino acids, peptides, proteins, lipids organic compounds, aromatic compounds, salts, metabolites, waste compounds, cyclic nucleotides, anions, cations, hydroxyl ions, acid, carbonate ions, plant extracts, insect extracts, nematode extracts and microbial extracts.

According to still another embodiment, the invention relates to any of the devices described above, wherein said permanent compound and/or agent changes the texture of the first surface of said solid support. Preferably, said agent and/or permanent compound is a lubricant.

According to another embodiment, the invention relates to any of the devices described above, comprising a condition, wherein said condition is an energy source selected from the group consisting of sources providing an electric field, a magnetic field, ultrasonic waves, high energy waves like laser beams; sources of thermal energy providing heat or cold; and sources of radiation; or a combination of at least two of such energy sources.

The invention further relates to any of the devices described above, wherein the surface of said porous support supports growth and/or breeding of the living organisms. Preferably said porous support is non-invasive.

According to another embodiment, the invention relates to any of the devices described above, wherein said behavioural barrier delineates test areas or test arrays on and/or within the solid porous support.

The invention also relates to any of the devices described above, further characterized in that said solid porous support comprises at least one effector molecule. In one embodiment, said at least one effector molecule is printed on the porous support. In another embodiment said at least one effector molecule is comprised within the pores of the porous support. In a further embodiment, said at least one effector molecule is comprised within predefined regions of the porous support.

The invention also relates to any of the devices described above, wherein said effector molecule is chosen from the group consisting of nutrients, enzyme substrates, test compounds, inducer molecules, chaperone proteins, hormones, oligopeptides, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, and drugs selected from a chemical or natural drug candidate library, or modified analogues of any of said molecules, or any combination of said molecules.

The invention also relates to any of the devices described above, further characterized in that said solid porous support comprises nutrient molecules and/or other compounds designed to maintain the organism in an appropriate state. According to one embodiment, said nutrient molecules and/or other compounds are printed onto the porous support. According to another embodiment, said nutrient molecules or said other compounds are comprised within the pores of the porous support.

The invention more specifically relates to any of the devices described above wherein said solid support is a metal oxide solid support, preferably an aluminium oxide solid support.

The invention more specifically relates to any of the devices described above wherein said support is a flow-through solid support.

The invention also relates to any of the devices described above, further comprising a supply chamber in contact with the second surface of said solid support. Preferably, said supply chamber comprises at least 1 compartment. Preferably, said at least one compartment is provided with a liquid medium comprising at least one effector molecule.

According to a more specific embodiment, the invention relates to any of the devices described above, wherein said at least one compartment is provided with a liquid medium comprising a gradient of at least one effector molecule, more preferably said liquid medium comprises a 2D gradient of at least two effector molecules.

The invention further relates to any of the devices described above wherein the number of compartments of said supply chamber is equal to the number of predefined regions on the porous support.

According to a preferred embodiment, the invention relates to any of the devices described above wherein said effector molecule is chosen from the group consisting of nutrients, enzyme substrates, test compounds, inducer molecules, chaperone proteins, hormones, oligopeptides, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, and drugs selected from a chemical or natural drug candidate library, or modified analogues of any of said molecules, or any combination of said molecules.

Preferably, the invention relates to any of the devices described above, wherein said supply chamber is in liquid contact with said second surface of said solid support.

More specific, the invention relates to any of the devices described above wherein the said at least one effector molecule is transported passively or actively through said porous support. Alternatively, said at least one effector molecule diffuses through said porous support to the cellular components by contact force. According to yet another alternative embodiment of the present invention, said at least one effector molecule is transported actively through said porous support by pumping, magnetically, electrically, or by piezo-electronic force.

According to another embodiment, the invention relates to any of the devices described above, further characterized in that the first surface is coated with poly-L-lysine as described in the International patent application WO 20041089531 which content is herein incorporated. In a more specific embodiment, the invention relates to the use said device for supporting growth of nematodes.

In a further embodiment, the devices of the invention further comprise at least one living organism chosen from the group comprising nematodes, Dictyostelium discoideum, colonial gliding bacteria including Myxobacter xanthus, bacteria capable of moving over solid surfaces, Drosophila melanogaster and other insects assuming that any ability to jump or fly is disabled if normally present (e.g. wingless mutants of Drosophila), slime moulds, protozoa such as amoeba, tissue culture cells capable of migration derived from larger organisms, motile spores and gametes.

According to yet another embodiment, the invention relates to a method for producing any of the devices described herein comprising:
printing or placing an agent and/or a condition on the first surface of the porous support delineating behavioural and/or physical barriers, wherein said barriers are printed on the first surface of the porous support so that it is drawn into the porous support and as such, completely or in part comprised within the pores of the porous support, therewith forming a three-dimensional compartmentalization of the porous support, wherein said agent and/or a condition is optionally mixed with a permanent compound,
optionally printing or placing effector compounds on the first surface,
optionally printing or placing nutrient sources on the first surface,
optionally inoculating the device with living organisms,
optionally contacting the second surface with a supply chamber for effector molecules, and/or
optionally contacting the second surface with a supply chamber for nutrients.

The invention further relates to a method for sensing behaviour and/or motility of motile living organisms in a multiplexed/microarray format comprising:
providing a device of the present invention,
inoculating the device with living organisms, and
detecting and/or identifying and/or characterizing a phenotypic or behavioural change, or change in activity in said organism(s) and/or in the offspring of said organism(s).

In another embodiment the invention relates to a method for screening test/effector molecules which affect behaviour and/or motility and/or health of a motile living organism in a multiplexed/microarray format comprising:
providing a device of the present invention,
inoculating the device with living organisms, and
detecting and/or identifying and/or characterizing a phenotypic, behavioural or biochemical change induced by said test/effector molecules in said organism(s) and/or in the offspring of said organism(s).

The invention further relates to a method for screening test/effector molecules which affect behaviour and/or motility and/or health of a motile living organism in a multiplexed/microarray format comprising:
providing a device of the present invention,
inoculating the device with living organisms,
delivering at least one effector from above the support by a means chosen from the group consisting of a delivery mask, a microfluidics device, a high precision x-y-z micro-pipettor, inkjet printer, and manual handling, and
detecting and/or identifying and/or characterizing a phenotypic, behavioural or biochemical change induced by said test/effector molecules in said organism and/or in the offspring of said organism.

Preferably, said motile living organisms referred to in the methods described above are selected from the group consisting of nematodes, Dictyostelium discoideum, colonial gliding bacteria including Myxobacter xanthus, bacteria capable of moving over solid surfaces, Drosophila melanogaster and other insects assuming that any ability to jump or fly is disabled if normally present (e.g. wingless mutants of Drosophila), slime moulds, protozoa such as amoeba, tissue culture cells capable of migration derived from larger organisms, motile spores and gametes.

According to yet another preferred embodiment, said (motile) living organism(s) used in the methods described above, is (are) fluorescently or luminescently labelled, labelled with small radio transmitters or radioactive tags or wherein said organism is coloured or labelled enabling thermal tracking. More preferably, said living organism(s) remaining within one predefined region is (are) differentially (differently) labelled, coloured or coded.

The invention further relates to any of the methods described above wherein said detection and/or identification and/or characterization is performed in real-time; alternatively, said detection and/or identification and/or characterization is performed in an end-point format.

According to a specific embodiment the invention relates to any of the methods described above wherein said detection and/or identification and/or characterization of phenotypic, behavioural or biochemical changes or change in organism number is performed by a method chosen from the group consisting of light microscopy, electron microscopy, luminescence, fluorescence.

The present invention further relates to the use of any of the devices described herein for testing repelling compounds from a library of compounds.

The present invention also relates to the use of any of the device described herein for testing attractants from a library of compounds.

The present invention also relates to the use of any of the devices described herein for studying behaviour and/or motility of living motile organisms and/or their offspring.

The present invention further relates to the use of any of the devices described herein for functional screening of phenotypic, behavioural, health and/or motility responses of a living organism and/or their offspring in response to a test/effector molecule.

According to yet another embodiment, the present invention relates to a kit for performing any of the methods herein described comprising any of the devices of the present invention, and, optionally, further comprising living motile organisms.

Additional features and advantages of the invention will be set forth in the detailed description which follows, and in part will be apparent from the description, or may be learned by practice of the invention. The objectives and other advantages of the invention will be realized and attained by the embodiments particularly pointed out in the detailed description and appended claims.

### DETAILED DESCRIPTION OF THE INVENTION

Before the devices and methods of the present invention are described, it is to be understood that this invention is not limited to particular methods, components, or devices described, as such methods, components, and devices may, of course, vary. It is also to be understood that the terminology used herein is not intended to be limiting the invention.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The present invention provides a system for high-throughput screening that is automation-friendly and allows parallel processing of numerous studies or tests.

The present invention provides a device comprising a solid support having first and second surfaces suitable for maintaining motile living organisms of any suitable size.

The nature and geometry of the device will depend upon a variety of factors. The size of the device may depend on the size and/or the number of the organisms deposited thereon and/or may depend on the type of study preformed and/or the number of compounds to be screened. In other words, there are not limits to the size of the device except for its intended use.

Devices according to the present invention comprise an array of test areas wherein the bottom of each test area is a solid porous support having first and second surfaces and at least one area with a plurality of through-going channels. Each porous solid support in a test area may further comprise a microarray or an array of micro-spots. The present invention therefore also relates to an array of arrays. It is understood by the term “test area” that these represent areas of the array which are directly involved in the analysis of test compounds or other reactants or living organisms or samples onto the solid support(s). Said device or said test areas may have any suitable shape including without limitation circular shape, square shape, rectangular shape and the like.

For instance the device may be as small as a typical microarry which is the size of a glass microscope size (75×25 mm) or smaller. Also devices comparable to 384 or 96 well plate sized arrays (120×80 mm) are suitable for the purposes of the invention. The critical aspect of the device is that it allows highly multiplexed parallel testing on surface test areas. Therefore, even larger arrays are envisaged if there is the need to have either larger test areas or to perform larger tests, i.e. larger devices will still be divided into multiple test areas. Therefore, when used in the present invention, the terms “device”, “array” or “microarray” are used interchangeable and are not intended to correspond with particular or preferred sizes of the device.

While it is known in the art to grow cells or monolayers of cells on solid supports, for instance as described in U.S. Pat. No. 6,103,479, for high throughput screening of compounds, these supports however are not suitable for testing motile organisms. One of the problems is how the motile organisms could be maintained in a substantially normal behaviour in the test areas without being physically immobilized.

The present invention solves this problem by providing a device for testing compounds in a highly multiplexed array format for motile living organisms without physically immobilizing, i.e. attaching by any possible means, the organisms to the first surface of the support. To achieve this, the first surface comprises an agent and/or condition delineating behavioural and/or physical barriers for motile living organisms. Said behavioural barriers according to the present invention are adapted for sensing by said living organism. The term “sensing” means to perceive by the senses, for instance by sight, tasting, hearing, smelling, touching, feeling, etc. Further, said “behavioural” barriers may also be named “chemical” barriers because they may incorporate a chemical compound. In this respect, “chemical” compound also encompasses the repellents and attractants described further. In addition “behavioural” barriers also encompass “non-chemical” barriers such as electromagnetic, electrical, light and heat barriers and barriers comprising some forms of radiation or magnetism or barriers comprising ultrasonic waves or high energy waves like laser beams.

A “physical” barrier according to the invention relates to any barrier which provides a 2 dimensional or 3 dimensional compartmentalization of the device and/or the first surface and/or the solid support and which prevents the organisms to pass physically, i.e. because it is an obstacle (obstruction/hindrance), which impedes action or progress or further movement of the organism in the direction it was going. According to the invention, a physical barrier may also constitute a behavioural barrier: said physical barriers may also be adapted for sensing, for instance by sight, touch etc. A critical aspect of the invention is that said behavioural and/or physical barrier forces or induces the living organisms to remain localized within a predefined region of said support without applying any physical act or power on the organism itself. The barriers are intended to prohibit the motile organisms to pass the barriers and/or leave the predefined regions.

In one aspect, the barriers are printed or deposited or placed on the first surface of the support by a means chosen from, but not limited to, the group comprising a high precision x-y-z pipettor, inkjet printer, photolithography, contact printing and manual handling, or by any other means described further.

Preferably, the barrier is substantially flat, level or almost so with the first surface of the device. This has certain handling or imaging advantages, for instance a high-powered microscope can be easily focussed on the surface/device from above, which would be difficult with a walled barrier such as a multiwell plate format.

In another aspect of the invention, the barriers, or the agents or conditions constituting the barrier, are printed (or deposited or placed) on the first surface of the device so that it is drawn into the porous support and as such, completely or in part comprised within the pores of the porous support. According to other embodiments of the invention, the barriers pass from the first surface to the second surface of the support through the pores of the support. According to these embodiments, the barriers are forming a three-dimensional compartmentalization of the device; i.e. the porous support becomes compartmentalized by a non-porous barrier area or the barrier is surrounding the porous the support, i.e. the test areas in the solid support are surrounded by a barrier.

In some embodiments of the invention, instead of the term “barrier”, the term “mask” may also be used. A “mask” may form a behavioural and/or a physical barrier, wherein an agent may be mixed with a permanent compound or wherein a condition may be localized within a permanent compound. Accordingly, an agent or condition according to the invention delineates a behavioural or physical barrier or mask wherein said agent or condition may or may not be admixed wit a permanent compound. Said permanent compound usually relates to polymeric materials. Barriers or masks are placed or printed or deposited on a porous support and usually form a grid mask through the porous support. Where the agent or condition is admixed with a permanent compound, the barrier or grid which forms is said to be a solid grid mask through the porous support. Such grid mask on a solid support allows certain regions on said support to be exposed and other regions to remain covered and inaccessible to the analysis performed on the exposed regions. Said mask or barrier may thus form a (three-dimensional) grid of polymeric material through said porous support. The grid mask makes the support suitable for array analysis.

The mask may contain an agent or a condition according to the invention, admixed or not with a permanent compound. For example, on the one hand the permanent compound may be admixed with an agent wherein said agent ensures the motile organisms to remain within a test area on the porous support. On the other hand, the agent or condition delineating a behavioural or physical barrier may be provided on top of a test area on the porous support (islands), said test area becomes than surrounded by a permanent compound in the shape of grid mask which in itself does not comprise an agent or condition according to the invention. Alternatively, the test areas on the porous support as well as the surrounding grid may both contain an agent or a condition for keeping motile organisms confined at a predetermined test area.

According to one embodiment of the invention, said agent or condition is printed (or applied or deposited) directly onto the first surface of the support and not in admixture with another compound.

In other embodiments, the agent is mixed with a permanent compound, or the condition is comprised within a permanent compound which is printed (or applied or deposited) onto the first surface of the support.

Permanent compounds according to the invention may be of polymeric or non-polymeric origin. Examples of preferred polymers include natural or synthetic latex; plastics; rubber; resins; proteins or polypeptides, i.e. poly-lysine, polyurethane, collagen, fibronectin; carbohydrates; ethylene glycol, propylene glycol, oleyl-O-poly(ethylene glycol)-ether; or other polymers that form by drying (i.e. some glues, silane, siloxane). Examples of non-polymeric compounds include metal depositions, i.e. gold, copper or aluminium, magnetic particles, ink, dyes, etc.

According to the invention, the mixture of the permanent compound with the agent or condition can be deposited on the first surface by any of the means described earlier.

Another way of preparing a device comprising on its first surface and/or in the pores of the solid support a behavioural and/or physical barrier mixed with or comprised within a permanent compound suitable for array analysis is characterized in:
(i) providing a solid porous support suitable for array analysis having first and second surfaces, said solid porous support having channels extending from said first surface to said second surface;
(ii) depositing at a predefined area on said porous solid support a polymeric material to said first surface, said polymeric material comprising a solvent so as to temporarily decrease its viscosity and/or rate of polymerisation during the step of depositing;
(iii) allowing said polymeric material to enter said channels of said solid porous support;
(iv) removing said solvent by contacting said first surface with a wash solution and draining the solvent/wash solution through said channels so as to restore the polymerisation rate of the polymer material within said channels wherein a mask on said solid support is formed;
further characterized in that said agent or condition is added to (or mixed with) said polymeric material prior to or after the depositing of the polymer onto the first surface.

In this method, the use of natural as well as synthetic latex as permanent compound is contemplated. Non-limiting examples of suitable latex polymers include polyvinyl chloride (PVC), polyurethanes, silicones, synthetic latex such as acrylics, polyvinyl acetate, polystyrene, styrene acrylics, styrene butadiene, polyvinyl acetate, vinyl acetate-ethylene, vinyl acrylics. Other polymers that form by drying such as a number of glues may also have their polymerisation slowed by ethylene glycol or another solvent.

Suitable solvents for use in the above method include without limitation glycols and glycol ethers. Non-limiting examples of glycols are ethylene and propylene glycol. Non-limiting examples of glycol ethers, also known as cellosolves and known for their use in surface coatings, such as lacquers, paints, and varnishes; fingernail polishes and removers; dyes; writing inks; cleaners; and degreasers include ethylene glycol monoethyl ether, ethylene glycol monobutyl ether, propylene glycol monomethyl ether, dipropylene glycol monomethyl ether, dipropylene glycol dimethyl ether, tripropylene glycol monomethyl ether, and propylene glycol methyl ether acetate.

The present method provides for accurately masked (i.e. provided with barriers) solid supports. Fine resolution-printing is accomplished by dispensing the masking polymer material with printer heads or ink-jet nozzles onto the support's surface. The computer controlled dispensing mechanism allows a well-controlled placing of droplets onto the surface of the support which then form a network of horizontal and perpendicular lines which are usually uniformly spaced but depending on the application's requirements may form non-uniformly spaced lines as well.

Mask grids applied on the surface of a solid porous support according to the present invention allow the grid to be formed through the depth of the porous support. Advantageously, the geometry of the grid is identical to the geometry of the predefined regions wherein the organisms need to reside.

According to the invention, the barriers delineate test areas, i.e. predefined regions, wherein the living organisms reside. It should be understood that the form and size of the test areas is very versatile and adaptable to the needs of the investigator or to the needs of the organisms under study. The devices of the present invention are suitable and adaptable to any living and motile organism. Examples of organisms which are used in the present invention comprise, but are not limited to, (wild type or mutant or recombinant) nematodes, Dictyostelium discoideum, colonial gliding bacteria including Myxobacter xanthus, bacteria capable of moving over solid surfaces, Drosophila melanogaster and other insects assuming that any ability to jump or fly is disabled if normally present (e.g. wingless mutants of Drosophila), slime moulds, protozoa such as amoeba, tissue culture cells capable of migration derived from larger organisms, motile spores and gametes.

It should thus be clear that the size of the predefined region may be adapted to the size or the needs of the organism to be placed on the device. As a way of example, but not intended to limit the invention, in the following table an exemplary list of organisms and recommended sizes of test area is shown:

| Organism | Description | Simple test area |
|---|---|---|
| Myxococcus xanthus | Colonial | 100 micron to 20 mm square |
| | bacterium | |
| Dictystelim discoideum | Slime Mould | 100 micron to 20 mm square |
| C. elegans | Nematode | 200 micron to 40 mm square |
| Wingless Drosophila | Mutated | 2 mm to 80 mm square |
| | fruit fly | |

Whilst in some embodiments of the invention, simple compartments are envisaged, so are more complex compartments (e.g. mazes, labyrinths, etc.) contemplated in other embodiments of the invention. Accordingly, these more complex compartments might be larger.

According to other embodiments, the agents or conditions of the invention demark islands (as opposed to barriers) which could be refuges for the living organisms.

In one embodiment of the invention, the agent in the barriers is a repellent, said barrier surrounding or delineating predefined regions wherein the living organism needs to remain. Examples of repellents which are used in the present invention comprise, but are not limited to, hormones, detergents, amino acids, peptides, proteins, lipids organic compounds, aromatic compounds, salts, metabolites, waste compounds, cyclic nucleotides, anions, cations, hydroxyl ions, acid, carbonate ions, extracts from pathogens (i.e. pathogens of the organism on the support) plant extracts, insect extracts, nematode extracts and microbial extracts. For instance, preferred repellents for use with nematodes are sodium dodecyl sulphate, trehalose, cyclic nucleotides, anions, cations, hydroxyl ions, acid, some forms of carbonate ions or aromatic compounds. In addition, certain plant, insect or nematode extracts may repel head-defective nematode mutants, due to their altered chemotaxis. Further, Dictyostelium discoideum, which is a social amoeba, is known to be very sensitive to cyclic AMP levels. In another example, microbial extracts from the nematophagous fungi Drechmania coniospora or the bacterium Serratia marcescens are highly effective repellents for nematodes.

In other embodiments of the invention, said agent is an attractant which is printed (or deposited or placed) on the surface of the porous support, the barrier being the absence of an attractant rather than the presence of a repellent as in former embodiments of the invention. Preferably, said attractant is printed (or deposited or placed) on the surface of the predefined regions or islands wherein the organism needs to remain.

In other embodiments said agent, i.e. said attractant, is comprised within the pores of the porous support, as such delineating predefined regions or islands wherein the organism prefers to remain.

Examples of attractants for use with the devices of the invention comprise, but are not limited to, hormones, pheromones, detergents, nutrients including prey organisms or extracts thereof, amino acids, peptides, proteins, lipids organic compounds, aromatic compounds, salts, metabolites, waste compounds, cyclic nucleotides, anions, cations, hydroxyl ions, acid, carbonate ions, plant extracts, insect extracts, nematode extracts and microbial extracts. Preferably compounds that are hormones or pheromones, or nutrients are envisaged.

According to yet another embodiment the invention relates to a device as described above wherein the first surface comprises a condition, optionally incorporated in or as a part of a permanent compound, said condition delineating or marking a behavioural and/or a physical barrier wherein the living organism is forced to remain. Preferred conditions according to the invention comprise, but are not limited to, energy sources selected from the group comprising sources providing an electric field, a magnetic field, ultrasonic waves, high energy waves like laser beams, or sources of thermal energy providing heat or cold, sources of radiation, or a combination of at least two of such energy sources.

In specifically preferred embodiments, the devices of the invention comprise a barrier capable of delivering an electric shock or delivering localized illumination or delivering a temperature barrier so that the organism studied will not cross the barrier and will remain confined in a particular area.

In addition, the pores of the support are ideally adapted for use of specific agents or conditions. For instance in case of inducing an electrical or magnetic field which changes the behaviour of the organism, i.e. it will remain in predefined regions on the support, the pores are ideally suited to comprise a conducting or magnetic material, respectively. As a way of an example, for instance a nano-scale gold electrode may penetrate the pores from the one surface to the other.

According to yet other embodiments, the invention relates to a device as described above where the agent and/or the permanent compound changes the texture of the first surface.

It should be appreciated by the person skilled in the art that the change in texture of the first surface does not necessarily or inevitably implies a molecular change of the (surface of) the support.

Changing the texture of the surface may have a positive, i.e. attractive, or a negative, i.e. repellent, effect on the organism. For instance, comprised within the present invention is an agent or permanent compound which locally makes the surface slick, such as for instance lubricants. In this respect, especially lubricants that can be applied to a surface such as Teflon are contemplated in the present invention. Other agents which change the texture of the surface and which are not attractive for a motile living organism to cross over are certain metals, such as for instance copper. As described earlier, the agent (optionally mixed with a permanent compound) may create islands instead of forming barriers. For instance, islands may be formed where the pores are blocked which could be refuges from the repellents in the pores, or certain (empty) pores may be blocking off attractants present in other parts or pores of the support.

As a result of the behaviour and/or physical barrier(s), the first surface of the support becomes divided into test arrays or test areas allow studying motile living organisms in a multiplexed (micro) array format. Because the organisms are confined to a predefined region without being physically immobilized, characteristics such as motile behaviour may be studied. Moreover, the organisms may feed and reproduce if required. The compartmentalization of the first surface is highly flexible, allowing different areas to be created for analysis, including mazes and other complex patterns for behavioural studies, or different repellent stimuli used.

According to the invention, the devices of the Invention preferably comprise a porous solid support.

Generally, the support may be composed of any material which will permit culturing living organisms. A number of materials suitable for use as supports as used in the present invention have been described in the art. Exemplary suitable supports in the present invention comprise materials including acrylic, acrylamide, methylene-bis-acrylamide, dimethylaminopropylmethacrylamide, styrenemethyl methacrylate copolymers, ethylene/acrylic acid, acrylonitrile-butadienestyrene (ABS), ABS/polycarbonate, ABS/polysulfone, ABS/polyvinyl chloride, ethylene propylene, ethylene vinyl acetate (EVA), nitrocellulose, polycarylonitrile (PAN), polyacrylate, polycarbonate, polybutylene terephthalate (PBT), polyethylene terephthalate (PET), polyethylene (including low 10 density, linear low density, high density, cross-linked and ultra-high molecular weight grades), polypropylene homopolymer, polypropylene copolymers, polystyrene (including general purpose and high impact grades), polytetrafluoroethylene (PTFE), fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene is (PCTFE), polyethylene-chlorotrifluoroethylene (ECTFE), polyvinyl alcohol (PVA), silicon styreneacrylonitrile (SAN), styrene maleic anhydride (SMA), and glass. Further exemplary suitable substrates comprise mixtures of two or more of the above-mentioned materials.

Other preferred suitable materials for the manufacture of supports of the present invention include metal oxides. Metal oxides provide a support having both a high channel density and a high porosity, allowing high-density arrays comprising different first binding substances per unit of the surface for sample application. In addition, metal oxides are highly transparent for visible light. Metal oxides are relatively cheap substrates that do not require the use of any typical microfabrication technology and, that offer an improved control over the liquid distribution over the surface of the substrate, such as electrochemically manufactured metal oxide membranes. Metal oxide membranes having through-going, oriented channels may be manufactured through electrochemical etching of a metal sheet.

The kind of metal oxide is not especially limited. Metal oxides considered are, among others, oxides of zirconium, mullite, cordierite, titanium, zeolite or zeolite analog, tantalum, and aluminium, as well as alloys of two or more metal oxides and doped metal oxides and alloys containing metal oxides. Therefore, the devices of the present invention preferably comprise a metal oxide solid support, preferably an aluminium oxide solid support.

Metal oxide supports or membranes as employed in the methods of the present invention may be anodic oxide films. As well known in the art, aluminium metal may be anodized in an electrolyte to produce an anodic oxide film. The anodization process results in a system of larger pores extending from one face and interconnects with a system of smaller pores extending in from the other face. Pore size is determined by the minimum diameters of the smaller pores, while flow rates are determined largely by the length of the smaller pores, which can be made very short. Accordingly, such membranes may have oriented through-going partially branched channels with well-controlled diameter and useful chemical surface properties. Useful thicknesses of the metal oxide supports or membranes as employed in the methods and apparatuses of the present invention may for instance range from 50 μm to 2000 μm (including thicknesses of 60, 70, 80, 90, 100, 110, 120, 130, 140, 200, 300, 400, 500, 1000, 1500 and 2000 μm). A particular suitable example of a support thickness is 60 μm.

A suitable support pore diameter ranges from 10 nm to 20 μm including 10 nm, 20 nm, 30 nm, 40 nm, 50 nm, 80 nm, 100 nm, 200 nm, 300 nm, 400 nm, 500 nm, 1 μm, 2 μm, 3 μm, 5 μm, 10 μm, 15 μm and 20 μm. A particular suitable example of pore diameter is 200 nm. These dimensions are not to be construed as limiting the present invention.

Due to the characteristic porous structure of the solid supports according to the present invention minimal amounts of effector molecules or compounds may be deposited on its surface or within the pores while remaining accessible at an effective concentration to the organisms on top of its surface. Accordingly, the solid porous supports according to the present invention offer advantages in terms of minimal amounts of printed compound having an effect. This may be due to the pore structure of the solid support trapping compounds in close proximity to the organism on top of it.

Advantageously, metal oxide membranes as described herein are transparent, especially if wet, which allows for assays using various optical techniques. Such membranes preferably have oriented through-going channels with well-controlled diameter and useful chemical surface properties. These solid porous supports preferably are flow-through porous supports. WO 99/02266 which discloses the Anopore™ porous membrane or support is exemplary in this respect, and is incorporated by reference in the present invention.

The devices of the present invention thus support for a non-invasive maintaining, i.e. culturing, growing, rearing, breeding, etc., of motile living organisms. The porous support allows the surface to be sufficient wet to support growth but not flood and is inert in that it is not adversely affecting the organism. Further, it is impossible for the organism to invade (or dig into or burrow into) these porous supports of the invention (unlike gel supports such as agar). In the methods of the present inventions, the organisms are thus kept in a two dimensional environment which is more advantageous for study purposes than a three dimensional environment.

As will be appreciated by a person skilled in the art, established protocols are available for culturing and maintaining the diverse organisms, i.e. described earlier, on the solid porous support. Such protocols may require the use of specialized coatings and selective media to enable growth and/or breeding of the organisms. None of such protocols is precluded from use with the method of the present invention.

Therefore, in preferred embodiments, the first surface of the device may further comprise compounds or nutrient sources, i.e. nutrients or prey microorganisms, or deliver other stimuli to create a microarray or other environment for studying the organism. Such other compounds are for instance buffers to maintain the organism(s) in an appropriate/healthy state, or antibiotics to control unwanted infections.

In the present invention, nutrients or other compounds may be provided to the surface of the solid support from underneath or from above and through the pores of said solid support. Said nutrients and/or other compounds may be provided by diffusion to the surface of the solid support from underneath or from above and through the pores of said solid substrate.

These nutrients or other compounds may also be printed or deposited or placed from above on the first surface of the support using different means for depositing compounds as described earlier. According to other embodiments, the other compounds or nutrient sources are comprised within the pores of the porous substrate.

Preferably, the provision of the nutrients and other compounds to support the growth of the organisms is under aseptic conditions well-known in the art. Said aseptic conditions may be accomplished by working in a laminar flow bench or by placing a cover on the support; i.e. on the side of deposit or growth of the organisms.

The devices according to the invention are thus ideally suited to test effector molecules in a multiplexed format. An effector molecule may be any molecule which may induce an effect on or in the organism under study. It is understood within the meaning of the present invention that both terms “effector” and “effector molecule” may be included in the general common term “effectors”. Another term which may be used in particular embodiments of the present invention instead of “effector molecule(s)” is “test compound(s)”.

An effector is a variable component in the assay and not a common component of the array environment, i.e. not a universal component of the growth medium.

Table 1 lists a number of effector molecules that may be used in the devices and methods of the invention. In particular, Table 1 summarizes possible combinations of effectors (reactants) and other reactants that may be supplied from a supply chamber, that may be printed on the solid support at the start of the experiment or analysis or that may be added (i.e. other reactants) during the experiment or analysis.

The devices of the invention are ideally suited to test a series of effector molecules, such as the ones chosen from the group comprising, but not limited to, nutrients, enzyme substrates, test compounds, inducer molecules, chaperone proteins, hormones, oligopeptides including modified analogues thereof such as PNA's or LNA's, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, or drugs selected from a chemical or natural drug candidate library, or modified analogues of any of said molecules, or any combination of at least two of said effector molecules.

Effector molecules also include a source that may be complex, an undefined mixture or a cellular source and the cell can be grown with the motile test organism on-chip. For example, siRNA designed to affect nematode behaviour may be expressed in bacterial cells which are co-cultured on the substrate with the nematodes. A consequence of the nematode consuming these bacteria is a selective knock-down of the mRNA levels transcribed from a specific target gene.

It should be clear that nutrients, provided (or printed or placed) on or in the predefined regions of the device may serve several functions: for instance as a nutrient to feed the organism; as an attractant to keep the organism in the predefined region; as an effector molecule as an experimental variable. Other effector molecules listed above may also serve distinct functions in test assays.

The present invention further relates to the use of the devices of the invention in methods wherein test compounds, i.e. effector molecules, are drugs or any compounds which might become a drug. The number of possible test compounds runs into millions. Commercially available compound libraries including peptides, proteins, sugars, etc. may be obtained from, e.g., ArQule, Pharmacopeia, Graffinity, Panvera, and Oxford.

According to the invention, the effector molecules may be printed or placed or deposited onto the first surface from above by any of the means already described herein. In certain embodiments, each predefined region of the device comprises a different effector molecule. According to other embodiments, the predefined regions of the device comprise the same effector molecule, however, in a different concentration or in a different formulation. According to other embodiments, the predefined regions comprise one organism (and optionally also its offspring) in each of the predefined regions. In some embodiments, all organisms on a device belong to the same species; in other embodiments the organisms represent different mutants originating from a wild type organism or species; in other embodiments different species or different organisms are present in distinct predefined regions; in other embodiments organisms may vary by sex (e.g. mating studies); in still other embodiments, different species are present in a single predefined region (e.g. predator-prey studies). It should be clear that the devices of the invention are very versatile and can be inoculated with any living organism(s) according to the investigator's needs or as required for the intended study.

According to other embodiments of the invention, the effector molecules may be present in the pores of the support or may be provided from the second surface of the support, i.e. opposite to the first surface and in contact thereto through the pores.

Delivery of effector molecules, nutrients or other compounds to the predefined regions on the support in order to keep the organism(s) in an appropriate/healthy state may be accomplished by using a liquid handling device but may equally be accomplished by manual handling.

Accordingly, a liquid handling device may be positioned on the substrate, wherein said liquid handling device may be a high precision x-y-z pipettor or inkjet printer containing one or more channels through which liquid can be dispensed, sequentially or in parallel, to positions corresponding to arrayed organisms/predefined region on the surface of the solid support. Alternatively, a superposing mask comprising transversal holes may be superposed onto the support, wherein said superposing is such that each transversal hole in said mask corresponds to an arrayed organism/predefined region on the surface of said solid support.

Alternatively, the effector molecules, nutrients or other compounds may also be delivered or spotted through ink-jet printing technology, a non-contact technology in which compounds (i.e. effector molecules, nutrients and the like) are sprayed onto the surface using technology adapted from computer ink-jet printers. The ink-jet method is sometimes called indirect because the compounds are sprayed onto the surface rather than being directly placed. Ink-jet methods may be capable of producing smaller spots, and because they avoid physical contact with the surface may prove to be more suitable, especially in the present invention when the organisms are already present on the support.

Useful ink-jet printing methodologies may include continues and drop-on-demand ink-jet methods. Most suitable ink-jet printing methods are drop-on-demand ink-jet methods, examples of which include piezoelectric and electrostatic ink-jet systems.

Further useful in the present invention are spotting robots or liquid handling devices. Most spotting robots or liquid handling devices use an X-Y-Z robot arm (one that can move in three dimensions) mounted on an anti-vibration table. Said arm may hold nozzles in case of non-contact spotting. In contact spotting, said arm may hold pins. Nozzles or pins are dipped into a first microtiter plate (or a plate having the same geometry or organisation of predefined regions as the solid porous support where the organisms reside) to pick up the fluid (e.g. test compound solution) to be delivered. The tips in case of pins are then moved to the solid support surface and allowed to touch the surface only minimally; the test compound solution is then transferred.

According to another embodiment of the invention, the device may further comprise a supply chamber, in contact with the second surface of the porous support for delivery of effector molecules, or nutrients or other compounds.

As will be well appreciated, a supply chamber as provided within the present invention allows the delivery of effector molecules or nutrients or other compounds to the solid support which otherwise may suffer impracticalities; e.g. which may clog the capillaries of e.g. a spotting device.

Depending on the assay which is envisaged, a supply chamber according to the present invention may be positioned towards the first or the second surface of the solid support. However, from a practical point of view this may be incompatible with the living organisms on the first surface; a supply chamber is then preferably contacted only with the second surface.

A supply chamber as provided with the present invention gives access of its content to at least one predefined region on the first surface of the solid support. Preferably, the supply chamber and/or any compartments in the supply chamber have the same geometry of the device and/or of the test areas defined by the behaviour and/or physical barriers described earlier. The supply chamber is attached to the support by either mechanical attachment (e.g. click on system or other), physical attachment or merely by being in liquid contact with the support. Physical attachment of the supply chamber to the solid support may be, by way of example and not limitation, thermal bonding, laser welding, ultrasonic welding, latex masking agents, glues or chemical welding (chemical solvent-based bonding). A washing step usually follows to remove any possible toxic product that may be derived from the attachment procedure. Said physical and/or liquid contact may not be permanent and as such allows subsequent supply chambers with diverse or equal contents to be combined with a same solid porous support. A removable supply chamber according to the invention offers the advantage and flexibility of transferring effectors to the organisms on the solid support and immediate interruption of said supply by removal of the chamber.

Accordingly, in one embodiment of the present invention, devices are provided, wherein a supply chamber is in liquid contact with the first and/or second surface of the solid support.

Liquid contact may be simply by orienting a supply chamber to the surface of the solid support that is opposite to the surface carrying the organisms. The capillaries within said solid support may draw the liquid into them from the underneath supply chamber. Alternatively, the solid support may simply rest on a liquid reservoir such as a dialysis membrane filled with liquid.

Non-limiting examples of supply chambers that may be in liquid contact with a solid porous support according to the present invention include gel patches and open capillaries that contact the porous solid support.

Physical attachment may be by resting the solid support on a solid matrix such as a gel or other porous support from which fluid is drawn. Physical attachment may provide structural support to the device.

Both liquid contact and solid attachment does not exclude the solid support as being part of the structure of the device in its entirety.

A supply chamber according to the present invention comprises a planar square, rectangular or circular surface and four upstanding walls surrounding the circumference of said surface to form a chamber having an open top and a closed bottom surface. The open (top) end of the supply chamber is oriented towards the first or the second surface of the solid porous support to which it becomes then physically attached or by liquid contact with the array. Useful supply chambers may also have open top and bottom surfaces.

The present invention thus also relates to a device as described earlier comprising a solid porous support, said support being at its first and/or second surface in liquid or in gaseous contact with a supply chamber or wherein said supply chamber is physically attached thereto

In fact, the structure of the supply chamber may be in physical contact with the solid support; however; in particular the porous support draws liquid into the capillaries or pores, even if the liquid comes from a gel (e.g. agar) permeated with e.g. nutrients and other compounds—as such liquid contact may be critical. Alternatively, a supply chamber according to the present invention may be attached to the porous solid support by gaseous contact; e.g. biogas sniffers.

A supply chamber according to the present invention may comprise multiple-use-insertions for parallel studies. Multiple-use-insertions are fixed, or optionally movable, separations allowing the supply chamber to be compartmentalized. The spatial organization of the inserts determines the number of compartments and the number of test areas covered by one compartment. If no inserts are used, the supply chamber is likely to comprise one compartment. The geometry of the inserts can be easily adapted to the geometry of the test areas or predefined regions on the first surface of the device. One compartment may cover one or more such predefined regions.

A supply chamber without an insert and hence a single compartment is particularly useful when a single effector or a single mixture of effectors or a gradient of one or more effectors is to be supplied towards the porous solid support. Two-dimensional gradients in particular offer to each position on the porous solid support a unique environment. Alternatively, multiple compartments may be present each with their own gradient of effectors.

Accordingly, in one embodiment of the present invention, devices are provided wherein said supply chamber comprises at least 1 compartment; i.e. 2, 3, 4, 5, 6, 7, 8, 9, 10 or even more compartments.

The number of compartments may be limited to the number of predefined regions on the solid support. However, larger predefined regions may be served by more than one compartment. In case the organisms need more than one effector molecule or nutrient or other molecule, more than one compartment may serve a single predefined region. The number of compartments in a supply chamber may also be limited according to the manufacturing of the device.

In another embodiment of the invention, a supply chamber is provided, wherein said at least one or more effectors are contained within a gaseous or liquid medium.

The at least one effector or effector molecule transported towards the porous solid support via the supply chamber may be contained within a solid, liquid or gaseous medium depending on the nature of the effector. For example, nutrients to induce and/or maintain growth of the organisms on the first surface of the porous solid support will usually be contained in an appropriate medium and provided from a supply chamber oriented with the open end towards the opposite outer second surface. The medium is typically provided as a liquid or gel medium including e.g. nutrients. It will be appreciated by the man skilled in the art that suitable media and nutrients for maintaining the respectively described organisms on the first surface, are available from commercial suppliers or may be prepared according to published recipes. The media are prepared using procedures known in the art.

In a further embodiment, devices are provided, wherein the at least one compartment of the supply chamber is provided with a liquid medium comprising a gradient of at least one effector molecule, in other embodiments a liquid medium comprising a 2D gradient of at least two effector molecules, e.g. 3, 4, 5, 6, 7, 8, 9, 10 or more effector molecules.

2D gradients in more than one compartment of a supply chamber may comprise an equal composition of effector molecules, said effector molecules in each compartment may differ in concentration or not.

The complexity may depend on the nature of the medium, i.e. for example a serum may present a very complex mixture of effectors. A mixture of effectors may also be accomplished by manual preparation; in this case the amount of each effector in said mixture is exactly known.

A supply chamber according to the present invention may comprise fixed inserts to form a supply chamber with a fixed number of spatially arranged compartments.

The reversibility of supply chamber attachment allows removal of a nutrient layer that may interfere with an assay due to for example auto-fluorescence. The removable supply chamber also permits sequential addition of effectors or gradients of effectors.

If a number of porous solid supports in the array of arrays need to be excluded from delivery of effectors via the supply chamber, than this may be achieved simply by leaving the corresponding compartments empty or by blocking them for any material transfer.

The supply chamber according to the present invention may be manufactured from materials well known in the art and suitable for receiving and storing of biological material such as metals including stainless steel and alloys, glass and plastics polymers. These materials preferable have a good chemical resistance, have stable physical properties, may be rigid, semi rigid or flexible and may exhibit any degree of translucence or opaqueness depending on the material stored within the supply chamber. Any materials that can be coated or chemically modified are suitable as well. Suitable materials are further preferably anti-fluorescent and do not allow the volume on the compartment(s) to change during the analysis. Plastics are particularly suitable materials for the manufacture of supply chambers according to the invention and may include natural polymers such as e.g. latex as well as chemically modified polymers such as e.g. vulcanized rubber and bakelite. Non-limiting examples of plastics for manufacture of supply chambers according to the invention include polyethylene terephthalate (PET, PETE), high density polyethylene (HDPE), polyvinyl chloride or PVC, low density polyethylene (LDPE), polypropylene, polystyrene, liquid crystal polymers (LCP), Topas® including combinations thereof.

According to the methods of the present invention, a supply chamber as described within the present specification allows access to the solid porous support of effectors or other reactants by either diffusion or active transfer.

Liquid contact of the supply chamber with the solid porous support allows diffusion of the effectors from the supply chamber through the porous solid support. Further, effectors may be passively transported by capillary action, by osmotic action, by liquid contact force or by convection. The term “contact force” as used within this specification means a direct surface contact between the solid porous support and the means for delivery of effectors or other reactants such as a supply chamber. Surface contact related to the supply chamber may be by the liquid surface of the medium within the chamber.

Active transfer of effectors from a supply chamber may be for example by pumping (both pushing and drawing), acoustic wave, by application of a low pressure above the solid support, or by vapour contact.

Effectors that may be provided by other means than supply chamber or liquid handling apparatuses include for example electromagnetic treatments, temperature treatment, pressure treatment and the like.

Multiplexity of analysis provided by the methods of the present invention is at multiple levels including (a) supply of nutrients or other compounds or effectors at the first and/or second surface of the solid support, (b) positionally directed supply to one or more test areas of at least one effector from the supply chamber, and (c) provision and storage of effectors, nutrients or other compounds within the porous structure of the solid support prior to assay performance.

The present invention further relates to a method for producing any of the devices described above comprising:
printing or placing an agent and/or a condition on the first surface of a porous support delineating behavioural and/or physical barriers, wherein said barriers are printed on the first surface of the porous support so that it is drawn into the porous support and as such, completely or in part comprised within the pores of the porous support, therewith forming a three-dimensional compartmentalization of the porous support, wherein said agent and/or a condition is optionally mixed with a permanent compound,
optionally printing or placing effector compounds on the first surface,
optionally printing or placing nutrient sources on the first surface,
optionally inoculating the device with living organisms,
optionally contacting the second surface with a supply chamber for effector molecules,
optionally contacting the second surface with a supply chamber for nutrients.

It should be clear that each of these method steps can be optional or obligatory, depending on which device is to be made or depending on the circumstances in which the device will be used. Also encompassed by the invention is any device obtainable by or obtained by any of the above methods for producing a device.

Since it may be desirable to provide test compounds on or in predefined regions within a porous support for screening at a later time-point, the present invention also relates to devices having test compounds immobilized on or comprised within (the pores of) the porous support. The devices described above are particularly suitable for the preservation of test compounds. The test compounds may be immobilized on the first surface of the support or provided in solution.

The use of compound libraries is particularly known to speed up drug discovery. Precipitation of some compounds is a recognized problem and known to occur with a large number of potent lead compounds. Due to the precipitation, often these compounds are excluded from screening programs because of the otherwise clogging of the liquid handling systems. A solution to this problem is provided by using a supply chamber in combination with the devices of the present invention. Large compound libraries may be stored within a multiplicity of supply chamber compartments, ready for use in the present assays.

Compound libraries may thus be stored in the supply chamber. They may be present in dry condition after e.g. slow evaporation or vacuum drying methods or e.g. by blowing air above and below the wells. Dried compounds can be dissolved later on when an assay needs to be performed. Alternatively, said compounds may be in solution already.

Depending on the solubility of the compound, diffusion may be total or partial and sufficient to allow for hit identification. Transfer of the compounds is not limited to diffusion, and may also be by pulsing a liquid sample back and forth through the porous support thereby maximising mixing of assay components. By pulsing a sample within the pores of the support, compounds in the supply chamber may be pulsed along.

Alternatively, compounds useful in the discovery process of drug candidates may be provided and stored within the porous structure of the solid support. Devices according to the present invention comprise a first surface with predefined regions, i.e. test areas, wherein the bottom of each area is a solid porous support with a plurality of through-going channels. Compounds may be dispensed into the channels and dried or concentrated into the porous support using e.g. slow evaporation or vacuum drying methods or by e.g. by blowing air or an inert gas such as e.g. helium above and below the support. These library plates may be stored until assay performance. Assays are directly performed in these compound plates by adding the appropriate buffers and further essential components. The use of these compound plates avoids laborious and time consuming compound distribution.

Accordingly, in one embodiment of the present invention a device is provided, wherein within its porous structure an array of test compounds, i.e. effector molecules, is provided in dried, lyophilized, gaseous or supercritical state.

In another embodiment, a device according to the present invention is provided comprising a porous solid support and a supply chamber, wherein an array of test compounds is provided within predefined regions on the surface of said support, said test compounds are in liquid, gaseous or supercritical state.

Said test compounds are usually not immobilized within said porous support. However, test compounds may be immobilized temporarily e.g. with triggered release (e.g. temperature, or laser activated release) or e.g. whilst still immobilized may have an effect on the organisms e.g. through surface interactions. Alternatively, compounds may be immobilized temporarily with a release that is susceptible to a specific cleaving agent either chemical or enzymatic such as for instance a nucleic acid sequence that contains the recognition site for a restriction endonuclease, or a specific peptide (or protein) that contains the cleavage site for the corresponding peptidase (or protease).

Test compounds may be immobilized within the porous structure of the solid support temporarily (e.g. to provide a defined release rate) or permanently wherein the permanently immobilized compounds may still have an effect on the organism, e.g. via external receptors or senses. Test compounds may also be immobilised within the supply chamber from where they may be delivered to the organisms after having first entered a gas or liquid phase.

According to other embodiments of the invention, the device is used for sensing or studying the behaviour or motility of one or more living motile organisms, such as the ones herein described. In order to distinguish or to mark individual organisms, the organisms may be labelled with a luminescent, preferably a fluorescent marker. Alternatively, the organisms may be provided with small radio transmitters or radioactive tags (or generally, emitters of any kind of radiation). The organisms may also be followed or studied by thermal tracking.

Means for detecting signals in general are well known to those of skill in the art. Thus, for example, radiolabels may be detected using photographic film or scintillation counters, fluorescent markers may be detected using a photodetector to detect emitted illumination, and colorimetric labels are detected by simply visualizing the coloured label. Further detection means are for example (micro-)calorimetry and (light)-microscopy.

Alternative to the labelling of the organisms themselves, it may be useful to detect excretion products, such as waste, slime trails, hormones, which could be also be detected by luminescence (or fluorescence). These excretion products, thus not only the organism itself, or other signs are indicative for their passage. To that effect, labelling molecules and/or detector molecules may be provided to the organisms for instance by mixing with nutrients.

Particularly useful fluorescent molecules include, by way of example and not limitation, fluorescein isothiocyanate (FITC), rhodamine, malachite green, Oregon green, Texas Red, Congo red, SybrGreen, phycoerythrin, allophycocyanin, 6-carboxyfluorescein (6-FAM), 2′,7′-dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5, Cy3), BODIPY dyes (e.g. BODIPY 630/650, Alexa542, etc), green fluorescent protein (GFP), blue fluorescent protein (BFP), yellow fluorescent protein (YFP), red fluorescent protein (RFP), and the like, (see, e.g., Molecular Probes, Eugene, Oreg., USA).

In addition, in some embodiments of the invention, the organisms are coded to differentiate individuals from each-other, such as by providing the individual organisms with a bar code, a spot, a mark, or the like.

In other embodiments, for instance where interaction between individual organisms are to be studied, it is more advantageous to study the transfer of an indicator from one organism to another, for instance to study interactions due to mating, predation or the like.

The present invention also relates to a device as described herein comprising at least one organism chosen from the group comprising, but are not limited to, (wild type or mutant or recombinant) nematodes, Dictyostelium discoideum, colonial gliding bacteria including Myxobacter xanthus, bacteria capable of moving over solid surfaces, Drosophila melanogaster and other insects assuming that any ability to jump or fly is disabled if normally present (e.g. wingless mutants of Drosophila), slime moulds, protozoa such as amoeba, tissue culture cells capable of migration derived from larger organisms, motile spores and gametes. Preferably, said device is kept or maintained under conditions favouring the viability of the organism.

Also comprised within the invention is a device as described above comprising at least one egg or larval of pupal stage of one of the organisms described herein, allowing, when placed on or in the appropriate conditions, the organism to leave the egg or to emerge from the larval or pupal stage at a self-chosen point in time.

The present invention also relates to a kit comprising any of the devices described herein, optionally comprising other compounds or nutrients to maintain and/or breed and/or rear living organisms and optionally comprising test compounds or effector molecules.

The distinct uses of the devices of the invention are well illustrated by the claims attached hereto. All of the references mentioned herein are incorporated by reference.

### EXAMPLES

### Example 1

### Screening Nematodes with a Combination of Drugs and siRNA

Nematodes worms are used as model organisms for screening processes relevant to the discovery of drugs relevant to human diseases, such as diabetes or psychiatric disorders. This example envisages a screening program in which a combination of drugs and siRNA delivery is used to screen for relevant changes in nematode behaviour.

A screening array is constructed in which 96 test areas are separated by either a physical or behavioural barrier. Each test area comprises porous Anopore™ substrate (Whatman, 60 μM thick with 0.2 μM diameter pores) treated with surface chemistries to suit the culture of both nematodes and their bacterial prey. In this example, test areas are either untreated or are coated with poly-L-lysine (latter may be advantageous for nematode growth). Test areas are then printed with effector molecules. In this case the effector molecules are members from a drug library that are predicted to treat a neuromuscular disorder in humans and therefore expected to affect the motility of nematodes. Alternatively or additionally, Escherichia coli bacteria expressing a specific siRNA that is designed to knock-down expression of a specific nematode mRNA may be inoculated into individual test areas. In this case, the siRNA will be delivered by the host bacteria being consumed by the nematodes. In this example, the siRNA component of the experiment is designed to target genes that may be relevant to drug metabolism and toxicity. Different combinations of a drug and an siRNA are used to screen for drugs with appropriate effects and toxicity. Screening array plates can be conveniently stored with the drugs and bacteria dried or freeze-dried and under vacuum until required.

When required, arrays are re-hydrated by placing them on Nematode Growth Medium agar containing appropriate supplements for nematode growth (e.g. cholesterol) and bacterial growth and siRNA induction (e.g. IPTG and antibiotics to maintain plasmids). Inoculation of each compartment is with 10-20 L4 stage nematodes expressing GFP either constitutively, or with the level acting as a reporter of viability. After inoculation the plates are incubated for 48 hours at 25° C. At 12 hour intervals each compartment of the array are imaged by rapidly taking a series of pictures at 1 second intervals for 2 minutes. Imaging is performed by low-powered fluorescence microscopy. Data is captured by a CCD camera and then analysed for organism number and development stage, motile behaviour reconstructing the images to track the movement and behaviour of individual nematodes. Behavioural analysis may also look at the association of nematodes, for example aggregation of individuals that can be indicative of starvation. Additional information may be provided by imaging organs within individual nematodes or recovering nematodes from specific test areas for further analysis such as by flow cytometry such as the COPAS system (Union BioMetrica). Alternatively, fixation and in situ hybridisation or recovery of nucleic acids can be performed without necessarily removing nematodes from the test areas.

A sample result of interest would be that a drug has the desired behavioural effect but is lethal if a cytochrome P450 gene is knocked down by siRNA. This may indicate an interesting drug candidate but one with possible detoxification issues.

TABLE 1
Summary of reactants that may be supplied from a supply chamber or that are printed on
the solid support at the start of an experiment or analysis. Some analysis may require
an additional provision of other reactants that are not yet provided at the start and that
may be added during the experiment or analysis. It is noted that all listed reactants may
be provided in all possible combinations, either simultaneously or sequential.

| From supply chamber | Printed on support | Other |
|---|---|---|
| Nutrients | Drugs or antibiotics or adjuncts to | Cellular component (or mixtures |
| | drug or antibiotics action (inhibitors, | thereof) |
| | cofactors) | |
| Buffers (osmotic or pH) | Nutrient supplements (e.g. individual | Preservatives, fixatives, agents to |
| | vitamins or amino acids) | stop enzymatic reactions, fluorescent |
| | | quenching agents |
| Enzyme substrates | Lyophilised cells | Vital dyes, tracers (e.g. radioactivity) |
| | | and other detection agents for cellular |
| | | function or visualization |
| Drugs or antibiotics | Natural products or derivatives | Light, radiation, other electromagnetic |
| | thereof | agents or temperature changes |
| Secreted compounds from | Proteins, nucleic acid, | Effectors, inducers |
| other organisms, e.g. | carbohydrates, and other | |
| hormones | macromolecules both natural and | |
| | synthetic analogues | |
| Vital dyes, tracers (e.g. | Scaffolds (complex protein or | Agonists, antagonists |
| radioactivity) and other | carbohydrate structures or synthetic | |
| detection agents for cellular | analogues) that affect cell growth or | |
| function or visualization | differentiation | |
| Viscous agents to regulate | Agents affecting adhesion of cells | Proteins, antibodies, antibody |
| the rate of fluid entry into the | (lectins, polylysine, antibodies, anti- | fragments, aptamers |
| substrate (e.g. glycerol) | adhesion agents) | |
| Detergents | Slow release agents for other | Toxins, mutagens |
| | compounds printed on the substrate | |
| Toxins, infectious agents, | Fluorescence to enable focusing | Lysing agents, washing liquids |
| mutagens | and monitoring of release of | |
| | compounds printed on the substrate. | |
| Preservatives, fixatives, | Toxins, infectious agents, mutagens | vectors, transfection agents |
| agents to stop enzymatic | | |
| reactions, fluorescent | | |
| quenching agents | | |
| Fractions from a fractionated | Fractions from a fractionated | |
| complex mixture | complex mixture | |
| Inducers (of gene expression, | Inducers (of gene expression, | |
| cellular processes, | cellular processes, pathologies, | |
| pathologies, differentiation) | differentiation) | |
| Proteins, nucleic acid, | | |
| carbohydrates, and other | | |
| macromolecules both natural | | |
| and synthetic analogues | | |
| Transfection reagents | | |
| Transcription factors | | |
| Agonists | | |
| Antagonists | | |

1. A device comprising a solid porous support having first and second surfaces, said first surface comprising an agent and/or condition delineating behavioural and/or physical barriers for motile living organisms, and, where behavioural, said barriers adapted for sensing by said living organisms and hence forcing said living organisms to remain localized within a predefined region of said support without said motile living organisms being physically immobilized on said support, wherein said barriers are printed on the first surface of said porous support so that it is drawn into the porous support and as such, completely or in part comprised within the pores of the porous support, therewith forming a three-dimensional compartmentalization of the porous support.

2. A device according to claim 1, wherein said agent is mixed with a permanent compound or wherein said condition is localized within a permanent compound, said permanent compound being printed or placed on said first surface and constituting a behavioural and/or physical barrier.

3. A device according to claim 2, wherein said permanent compound is a polymeric material containing at least one of the following: latex, rubber, plastic, resin, glue, protein or polypeptide or carbohydrate.

4. A device according to claim 2, wherein said permanent compound is a non-polymeric material.

5. A device according to claim 2, wherein said physical barrier is substantially flat.

6. A device according to claim 1, wherein said agent is a repellent, further characterized in that said agent is comprised within said barriers surrounding said predefined region wherein said organism needs to remain.

7. A device according to claim 6, wherein said agent is chosen from the group consisting of hormones, detergents, amino acids, peptides, proteins, lipids organic compounds, aromatic compounds, salts, metabolites, waste compounds, cyclic nucleotides, anions, cations, hydroxyl ions, acid, carbonate ions, extracts from pathogens, plant extracts, insect extracts, nematode extracts and microbial extracts.

8. A device according to claim 1, wherein said agent is an attractant further characterized in that said agent is comprised within the predefined region wherein said organism needs to remain.

9. A device according to claim 8, wherein said agent is chosen from the group consisting of hormones, pheromones, detergents, nutrients, prey organisms or extracts thereof, amino acids, peptides, proteins, lipids organic compounds, aromatic compounds, salts, metabolites, waste compounds, cyclic nucleotides, anions, cations, hydroxyl ions, acid, carbonate ions, plant extracts, insect extracts, nematode extracts and microbial extracts.

10. A device according to claim 1, wherein said permanent compound and/or agent changes the texture of the first surface of said solid support.

11. A device according to claim 10, wherein said agent or permanent compound is a lubricant.

12. A device according to claim 1, wherein said condition is an energy source selected from the group consisting of sources providing an electric field, a magnetic field, ultrasonic waves, high energy waves, laser beams; or sources of thermal energy providing heat or cold; and sources of radiation; or a combination of at least two of such energy sources.

13. A device according to claim 1, wherein the surface of said porous support supports growth and/or breeding of the living organisms.

14. A device according to claim 1, wherein said porous support is non-invasive.

15. A device according to claim 1, wherein said behavioural barrier delineates test areas or test arrays on and/or within the solid porous support.

16. A device according to claim 1, further characterized in that said solid porous support comprises at least one effecter molecule.

17. A device according to claim 16, wherein said at least one effecter molecule is printed on the porous support.

18. A device according to claim 16, wherein said at least one effecter molecule is comprised within the pores of the porous support.

19. A device according to claim 16, wherein said at least one effecter molecule is comprised within the predefined regions of the porous support.

20. The device according to claim 1, further characterized in that the first surface is coated with poly-L-lysine.

21. A device according to claim 17, wherein said effecter molecule is chosen from the group consisting of nutrients, enzyme substrates, test compounds, inducer molecules, chaperone proteins, hormones, oligopeptides, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, and drugs selected from a chemical or natural drug candidate library, or modified analogues of any of said molecules, or any combination of said molecules.

22. A device according to claim 1, further characterized in that said solid porous support comprises nutrient molecules and/or other compounds designed to maintain the organism in an appropriate state.

23. A device according to claim 22, wherein said nutrient molecules and/or other compounds are printed onto the porous support.

24. A device according to claim 23, wherein said nutrient molecules or said other compounds are comprised within the pores of the porous support.

25. The device according to claim 1, wherein said solid support is a metal oxide solid support.

26. The device according to claim 1, wherein said solid support is an aluminium oxide solid support.

27. A device according to claim 1, wherein said support is a flow-through solid support.

28. A device according to claim 27, further comprising a supply chamber in contact with the second surface of said solid support.

29. The device according to claim 28, wherein said supply chamber comprises at least one compartment.

30. The device according to claim 29, wherein said at least one compartment is provided with a liquid medium comprising at least one effecter molecule.

31. The device according to claim 29, wherein said at least one compartment is provided with a liquid medium comprising a gradient of at least one effecter molecule.

32. The device according to claim 29, wherein said at least one compartment is provided with a liquid medium comprising a 2D gradient of at least two effecter molecules.

33. The device according to claim 29, wherein the number of compartments of said supply chamber and the number of predefined regions on the first surface are equal.

34. The device according to claim 30, wherein said effecter molecule is chosen from the group consisting of nutrients, enzyme substrates, test compounds, inducer molecules, chaperone proteins, hormones, oligopeptides, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, and drugs selected from a chemical or natural drug candidate library, or modified analogues of any of said molecules, or any combination of said molecules.

35. The device according to claim 28, wherein said supply chamber is in liquid contact with said second surface of said solid support.

36. The device according to claim 30, wherein the said at least one effecter molecule is transported passively or actively through said porous support.

37. The device according to claim 30, wherein said at least one effecter molecule diffuses through said porous support to the cellular components by contact force.

38. The device according to claim 30, wherein said at least one effecter molecule is transported actively through said porous support by pumping, magnetically, electrically, or by piezo-electronic force.

39. The device according to claim 1, further comprising at least one living organism chosen from the group comprising nematodes, Dictyostelium discoideum, colonial gliding bacteria, Myxobacter xanthus, bacteria capable of moving over solid surfaces, Drosophila melanogaster and other insects assuming that any ability to jump or fly is disabled if normally present, wingless mutants of Drosophila, slime moulds, protozoa, amoeba, tissue culture cells capable of migration derived from larger organisms, motile spores and gametes.

40. A method for producing a device of claim 1 comprising:
printing or placing an agent and/or a condition on the first surface of the porous support delineating behavioural and/or physical barriers, wherein said barriers are printed on the first surface of the porous support so that it is drawn into the porous support and as such, completely or in part comprised within the pores of the porous support, therewith forming a three-dimensional compartmentalization of the porous support, wherein said agent and/or a condition is optionally mixed with a permanent compound,
optionally printing or placing effecter compounds on the first surface,
optionally printing or placing nutrient sources on the first surface,
optionally inoculating the device with living organisms,
optionally contacting the second surface with a supply chamber for effecter molecules, and/or
optionally contacting the second surface with a supply chamber for nutrients.

41. A method for sensing behaviour and/or motility of motile living organisms in a: multiplexed/microarray format comprising:
providing a device according to claim 1,
inoculating the device with living organisms, and
detecting and/or identifying and/or characterizing a phenotypic or behavioural change, or change in activity in said organism and/or in the offspring of the organism.

42. A method for screening test/effector molecules which affect behaviour and/or motility and/or health of a motile living organism in a multiplexed/microarray format comprising:
providing a device according to claim 1,
inoculating the device with living organisms, and
detecting and/or identifying and/or characterizing a phenotypic, behavioural or biochemical change induced by said test/effector molecules in said organism and/or in the offspring of the organism.

43. A method for screening test/effector molecules which affect behaviour and/or motility and/or health of a motile living organism in a multiplexed/microarray format comprising:
providing a device according to claim 1,
inoculating the device with living organisms,
delivering at least one effecter from above the support by a means chosen from the group consisting of a delivery mask, a microfluidics device, a high precision x-y-z micro-pipettor, inkjet printer, and manual handling, and
detecting and/or identifying and/or characterizing a phenotypic, behavioural or biochemical change induced by said test/Effector molecules in said organism and/or in the offspring of the organism.

44. The method according to claim 41, wherein said motile living organisms are selected from the group consisting of nematodes, Diclyostelium discoideum, colonial gliding bacteria, Myxobacter xanthus, bacteria capable of moving over solid surfaces, Drosophila melanogaster, and other insects assuming that any ability to jump or fly is disabled if normally present, wingless mutants of Drosophila, slime moulds, protozoa, amoeba, tissue culture cells capable of migration derived from larger organisms, motile spores and gametes.

45. The method according to claim 42, wherein said (motile) living organisms are fluorescently or luminescently labelled, labelled with small radio transmitters or radioactive tags or wherein said organisms are coloured or labelled enabling thermal tracking.

46. The method according to claim 45, wherein the living organisms within one predefined region are differentially labelled, coloured or coded.

47. The method according to claim 42, wherein said detection and/or identification and/or characterization is performed in real-time.

48. The method according to claim 42, wherein said detection and/or identification and/or characterization is performed in an end-point format.

49. A method according to claim 42, wherein said detection and/or identification and/or characterization of phenotypic, behavioural or biochemical changes or change in organism number is performed by a method chosen from the group consisting of light microscopy, electron microscopy, luminescence, fluorescence.

50-53. (canceled)

54. A kit comprising a device according to claim 1.

55-56. (canceled)
